(19) United States
(12) Patent Application Publication
(10) Pub. No.: US 2017/0367908 A1
(43) Pub. Date: Dec. 28, 2017

(54) Ecofriendly Diaper System

(71) Applicant: Kas Kasravi, West Bloomfield, MI (US)

(72) Inventor: Kas Kasravi, West Bloomfield, MI (US)

(21) Appl. No.: 15/632,362

(22) Filed: Jun. 25, 2017

Related U.S. Application Data
(60) Provisional application No. 62/354,747, filed on Jun. 25, 2016.

(51) Int. Cl. A61F 13/551; B65D 33/16; B65B 51/10; B65D 75/30; B65D 65/46

(52) U.S. Cl. CPC A61F 13/5511; B65D 75/30; B65D 65/46; A61F 13/5512; B65B 51/10; B65D 33/1616

(57) ABSTRACT

The present invention relates to an ecofriendly diaper system for children and adults. The core components that together make up the architecture of the system are: water-soluble diaper bag, washable diaper, optional moisture-resistant container and a heat-sealing element. Generally speaking, these components are structured such that the water-soluble diaper bags are stored in the moisture-resistant container, and dispensed when needed; a soiled washable diaper is placed in the water-soluble bag and sealed by a knot or by the heat-sealing element. The sealed water-soluble diaper bag containing the soiled diaper is stored. At the desired time, the stored water-soluble diaper bags are washed using a conventional household washer/dryer. The washed diapers are subsequently dried and stored at home. The water-soluble diaper bag is preferably made with polyvinyl alcohol film, where the film is optionally soluble at a temperature higher than the normal human body temperature, and made with an opaque color. The washable diaper is a conventional cloth diaper, and optionally externally coated with a thin film of moisture-proof material.

### PRIORITY STATUS

This application claims the benefit of U.S. Provisional Application No. 62/354,747, filed Jun. 25, 2016. The content of U.S. Provisional Application 62/354,747 is incorporated herein by reference for all purposes.

### BACKGROUND OF THE INVENTION

### 1. Field of the Invention

The present invention relates generally to diapers, and particularly to an ecofriendly diaper system.

### 2. Background

Currently, there are several solutions for storing, cleaning and disposing children and adult diapers. Some of these solutions attempt to provide disposable diapers, but these solutions fail to meet the needs of the industry because of high cost, the significant and undesirable contribution to landfills, and introduction of harmful chemicals to the users and the environment. An estimated 27.4 billion disposable diapers are used in the US, resulting in approximately 3.4 million tons of waste added to landfills each year. Disposable diapers often contain hazardous chemicals such as Dioxin, Tributyl-tin (TBT), and sodium polyacrylate. Other solutions attempt to provide washable cloth diapers, but these solutions are similarly unable to meet the needs of the industry because of foul odor and unsightliness of the soiled diapers, the high cost of cleaning by third-party services, and the possibility of not having diapers on hand when needed.

It would be desirable to have a solution that provides a low-cost and ecofriendly diaper system for storing, cleaning, and re-using diapers at home. Furthermore, it would also be desirable to have a material composition that is absent of harmful chemicals. Therefore, there has been a very long-felt need in the industry for a diaper management solution that provides for storage and cleaning of diapers at home, absent any of the aforementioned deficiencies.

### SUMMARY OF THE INVENTION

The present invention advantageously fills the aforementioned deficiencies by providing an ecofriendly diaper system, which provides a low-cost, environmentally friendly, and chemical-free solution for storing and cleaning soiled diapers at home. Ecofriendly is defined as having little, or no, harmful impact on the environment.

The present invention is an ecofriendly diaper system that is ecofriendly, and is capable of recycling soiled diapers at a low cost, without foul odor, without unsightliness, without harmful chemicals. The ecofriendly diaper system is comprised of a water-soluble diaper bag, which stores a soiled diaper, seals via a knot or heat, and can be stored and cleaned in a conventional manner at home. Preferably, the water-soluble diaper bag is made of a thin film of polyvinyl alcohol. Further, the polyvinyl alcohol film is manufactured to react with water at temperatures above the normal human body temperature, and optionally made with an opaque color. In an embodiment, the water-soluble bag is shaped long enough to permit sealing it via a knot. In a different embodiment, the water-soluble bag is sealed by heat. The sealed water-soluble bags containing soiled diapers are stored in a conventional household hamper, and cleaned using a conventional washer/dryer. The water-soluble diaper bags may be manufactured in different sizes to accommodate diapers of different of sizes.

The ecofriendly diaper system of the present invention optionally has a diaper container for storing and dispensing unused water-soluble diaper bags. This diaper container may have a moisture-proof lid to protect the unused water-soluble diaper bags in the diaper container. Further, the diaper container has an optional heating element to seal the water-soluble diaper bags containing soiled diapers. In the presence of a heating element, appropriate electrical safety measures are employed in compliance with the relevant safety and regulatory requirements. Further, either an ON/Off switch or an automated sensor is provided to heat and seal a water-soluble diaper bag when needed, absent the risk of overheating via a shut-off mechanism.

The water-soluble diaper bag is optionally manufactured with polyvinyl alcohol, an ecofriendly compound that has excellent film forming properties, is odorless and nontoxic, and has high tensile strength and flexibility. Further, the polyvinyl alcohol compound can be manufactured to dissolve in water at specific temperatures, such as one that is above the normal human body temperature, thus, avoiding accidental ruptures due to the moisture content of the soiled diapers. Still further, the polyvinyl alcohol compound may be manufactured in opaque and colorful finishes for aesthetic reasons and to hide the unsightliness of soiled diapers.

The conventional cloth or washable diapers are optionally externally coated by a thin film of moisture-proof material, such as plastic, to minimize leakage of the content through the walls of the diaper into the bag.

The present invention is unique when compared with other known systems and solutions, such as disposable or cloth diapers, in that it provides an ecofriendly diaper that does not contribute to landfills, does not contain any harmful chemicals, stores soiled diapers at home without foul odor or unsightliness, and can be washed in conventional household washer/dryer. The present invention is substantially more ecofriendly and cost effective compared to the currently used diaper solutions.

The present invention is superior to other systems and unique in that the overall architecture of the system is different from other known systems. More specifically, the present invention is unique due to the presence of: (1) a water-soluble diaper bag, which seals and stores soiled diapers at home without foul odor or unsightliness; (2) the ability to clean the soiled diapers in conventional household washer/dryer; (3) a container and dispenser for storing unused water-soluble diaper bags with a moisture-proof lid; (4) a heating element for sealing the water-soluble bags containing soiled diapers; (5) an ecofriendly system that does not contribute disposable diapers to landfills; (6) cost effective materials and processes; (8) water-soluble bags of different sizes, opacity, and colors; and (8) no harmful chemicals. The present invention fully recycles diapers in a convenient, safe, ecofriendly, and cost-effective manner.

Among other things, it is an object of the present invention to provide an ecofriendly diaper system that does not suffer from any of the problems or deficiencies associated with prior solutions.

The present invention now will be described more fully hereinafter with reference to the accompanying drawings, which are intended to be read in conjunction with both this summary, the detailed description, and any preferred and/or embodiments specifically discussed or otherwise disclosed. This invention may, however, be embodied in many different forms and should not be construed as limited to the embodiments set forth herein; rather, these embodiments are provided by way of illustration only and so that this disclosure will be thorough, complete and will fully convey the full scope of the present invention to those skilled in the art.

### BRIEF DESCRIPTION OF THE DRAWINGS

The features and advantages of the invention, as well as additional features and advantages thereof, will be more clearly understood hereinafter as a result of a detailed description of particular embodiments of the invention when taken in conjunction with the following drawings in which:

FIG. 1 illustrates the basic forms of a water-soluble diaper bag

FIG. 2 illustrates the sealed forms of water-soluble diapers bags

FIG. 3 illustrates the diaper container, optional lid, and heat-sealing element

FIG. 4 illustrates the overall system

### DETAILED DESCRIPTION OF THE INVENTION

The following discussion is directed to various embodiments of an ecofriendly diaper system. Although one or more of these embodiments may be discussed in detail, the embodiments disclosed should not be interpreted, or otherwise used, as limiting the scope of the disclosure, including any claims. In addition, one skilled in the art will understand that the following description has broad applications, and the discussion of any embodiment is meant only to be an example of that embodiment, and not intended to suggest that the scope of the disclosure, including the claims, is limited to that embodiment.

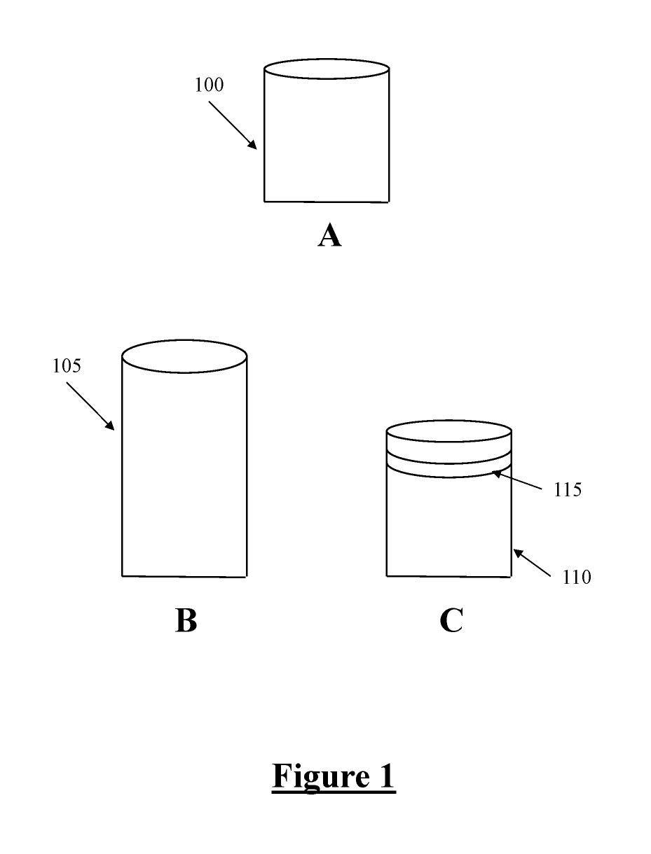

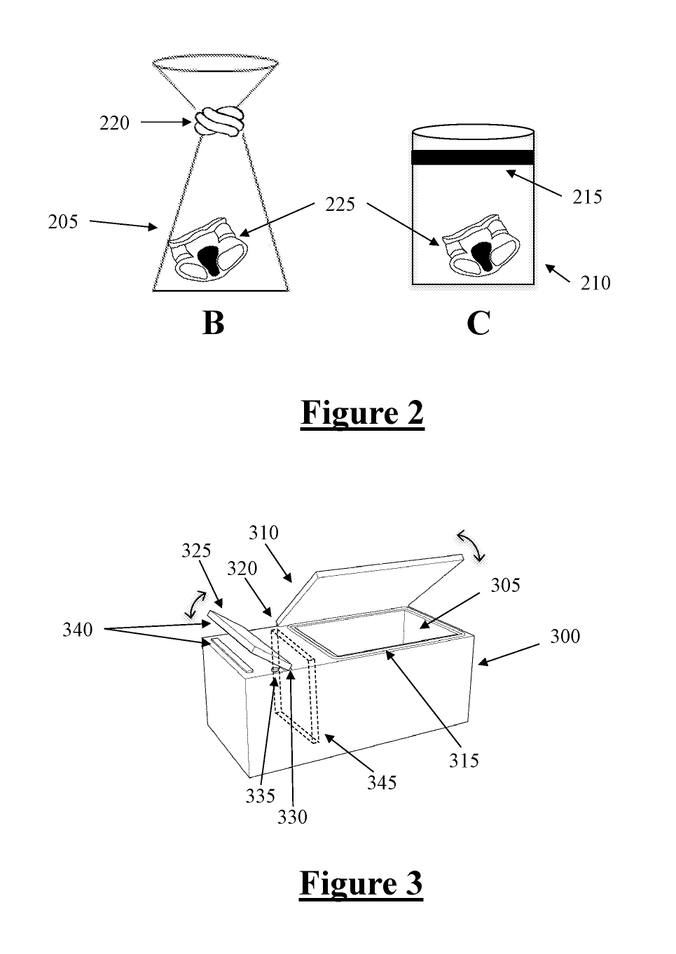

The figures herein follow a numbering convention in which the first digit or digits correspond to the drawing figure number and the remaining digits identify an element or component in the drawing. Similar elements or components between different figures may be identified using similar digits. For example, 145 may reference element “45” in FIG. 1, and a similar element may be referenced as 245 in FIG. 2. Elements shown in the various figures herein can be added, exchanged, and/or eliminated to provide a number of additional examples of the present disclosure. In addition, the proportion and the relative scale of the elements provided in the figures are intended to illustrate the examples of the present disclosure, and should not be taken in a limiting sense.

With reference to FIG. 1, the present invention comprises a water-soluble diaper bag 100, having one open end, used for storing soiled diapers. In the present invention, conventional and commercially available washable or cloth diapers are used. The water-soluble diaper bag 100 is manufactured either as a long water-soluble diaper bag 105, or a short water-soluble diaper bag 110 with a heat-sealing zone 115. In the preferred embodiment, the water-soluble diaper bag 100, 105, and 110 is preferably manufactured with polyvinyl alcohol film, which has excellent film forming, emulsifying and adhesive properties. Polyvinyl alcohol is also resistant to oil, grease and solvents. It is odorless and nontoxic and has high tensile strength and flexibility, as well as high oxygen and aroma barrier properties. However, these properties are dependent on humidity, in other words, with higher humidity more water is absorbed. The water, which acts as a plasticizer, will then reduce its tensile strength, but increases its elongation and tear strength. Polyvinyl alcohol is fully degradable and dissolves quickly in water at a pre-determined temperature.

FIG. 2 presents two methods for sealing a water-soluble diaper bag 100 containing a soiled diaper. The long water-soluble diaper bag 205 is sealed with a tight knot 220. The short water-soluble diaper bag 210 is sealed at the heat-sealing zone 215 with a heating element. A person of ordinary skill in the art would recognize that a variety of suitable heating elements are available for this purpose, and often comprise two heating elements heated by an electric current, and brought together in contact with a bag between them to seal the bag.

FIG. 3 presents an optional diaper container 300 for the storage of unused water-soluble diaper bags in a storage space 305. Due to the moisture reactiveness of the water-soluble diaper bag 100, the diaper container 300 comprises a storage area 305, a moisture-proof lid 310 and a moisture-proof-seal 315. Unused water-soluble diaper bags are stored in the storage space 305, and accessed via the moisture-proof lid 310, and then the lid is closed to protect the remaining bags in the storage space 305 from moisture. The moisture-proof lid 310 is configured to mate with the moisture-proof-seal 315 to seal the content of the storage area 305 when the lid is closed. In the preferred embodiment of the present invention, the moisture-proof lid 310 is pivotally attached to the diaper container 300 via a lid hinge 320, and snaps shut.

The diaper container 300 optionally provides a pair of heat-sealing elements 340 for sealing a water-soluble diaper bag containing a soiled diaper along the heat-sealing zone 115. The heat-sealing elements 340 are mounted on the diaper container 305 and against the inside surface of a heating element lid 325 in a mating manner when the heating element lid 325 is closed. In the preferred embodiment of the present invention, the heating element lid 325 is pivotally attached to a surface of the diaper container 300 via heating element lid hinge 330 as shown in FIG. 3.

The heat-sealing elements 340 are electrically powered to heat the elements and seal the water-soluble diaper bags containing diapers, using batteries or household electricity. In the presence of such heat-sealing feature, appropriate electrical safety measures are employed in compliance with the relevant safety and regulatory requirements. An ON/Off switch 335 activates the heating-sealing elements 340 long enough to seal a bag but not to overheat.

A thermal insulator 345 is positioned inside the diaper container 300 in between the heating sealing elements 340 and the storage space 305 to protect any unused diaper bags stored in the storage space. The thermal insulator is made of a commercially available thermal insulation material such as silicone, melamine Foam, polyimide foam, fiberglass, mineral wool, or polystyrene.

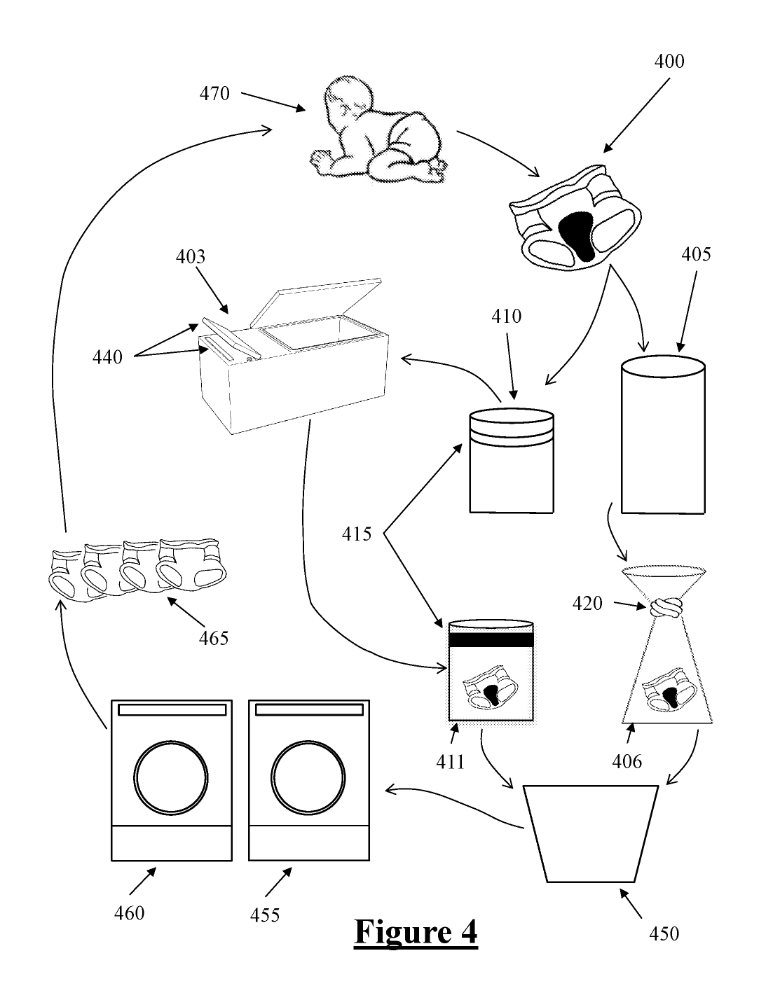

In its most complete version, the ecofriendly diaper system is made of the components illustrated in FIG. 4. Where, a soiled washable or cloth diaper 400 is placed inside the long water-soluble diaper bag 405 and sealed via knot 420, or a soiled washable or cloth diaper 400 is placed in the short water-soluble diaper bag 410 and sealed with the heat sealing elements 440, along the heat-sealing zone 415, provided in the diaper container 403. The unused water-soluble diaper bag 405 is stored in the diaper container 403. The sealed water-soluble diaper bags 406 and 411 are subsequently stored in a conventional household hamper 450. When desired, the water-soluble diaper bags containing soiled diapers may be cleaned in conventional household washer 455 and dryer 460. During the wash cycle of washer 455, the water-soluble diaper bags dissolve in the washer upon contact with warm water, the soiled diapers are washed in a conventional manner, providing clean diapers 465. The clean diapers 465 are stored at home until needed by a user 470. This process is repeated on a regular basis, as needed.

In a different embodiment of the present invention, the outer surfaces of the conventional washable or cloth diaper 400 are externally coated by a thin film of moisture-proof material, such as a thin film of plastic, to prevent leakage of the content through the walls of the diaper.

While the present invention has been described above in terms of specific embodiments, it is to be understood that the invention is not limited to these disclosed embodiments. Many modifications and other embodiments of the invention will come to mind of those skilled in the art to which this invention pertains, and which are intended to be and are covered by both this disclosure and the appended claims. It is indeed intended that the scope of the invention should be determined by proper interpretation and construction of the appended claims and their legal equivalents, as understood by those of skill in the art relying upon the disclosure in this specification and the attached drawings.

I claim:

1. An ecofriendly diaper system comprising:
a water-soluble diaper bag, having one open end, and sized to contain a soiled diaper.

2. The ecofriendly diaper system according to claim 1, wherein the water-soluble diaper bag is made with polyvinyl alcohol film.

3. The ecofriendly diaper system according to claim 2, wherein the water-soluble diaper bag has an opaque color.

4. The ecofriendly diaper system according to claim 3, wherein the water-soluble diaper bag is long, suitable for accommodating a knot close to the open end to seal the bag.

5. An ecofriendly diaper system comprising:
a diaper container;
a storage space within the diaper container, with one open, suitable for storing unused ecofriendly diaper bags;
a moisture-proof lid pivotally attached to a surface of the diaper container with a lid hinge, wherein the moisture-proof lid covers the storage space;
a moisture-proof seal along the gap between the storage space and the moisture-proof lid, wherein the moisture-proof seal seals the content of the storage space; and
a water-soluble diaper bag, having one open end, and sized to contain a soiled diaper.

6. The ecofriendly diaper system according to claim 5, wherein the water-soluble diaper bag is made with polyvinyl alcohol film.

7. The ecofriendly diaper system according to claim 6, wherein the water-soluble diaper bag has an opaque color.

8. The ecofriendly diaper system according to claim 7, wherein the water-soluble diaper bag is short, suitable for heat sealing.

9. The ecofriendly diaper system according to claim 8, wherein the water-soluble diaper bag has a heat-sealing zone close to the opening of the bag.

10. The ecofriendly diaper system according to claim 6, wherein a heating element lid is attached to a surface of the diaper container.

11. The ecofriendly diaper system according to claim 10, wherein the heating element lid is pivotally attached to the diaper container with a heating element lid hinge.

12. The ecofriendly diaper system according to claim 11, wherein a pair of heating elements are attached to a surface of the diaper container and a surface of the heating element lit in a mating manner, suitable for sealing the water-soluble diaper bag along the heat-sealing zone.

13. The ecofriendly diaper system according to claim 12, wherein an ON/Off switch activates the pair of heating elements.

14. The ecofriendly diaper system according to claim 13, wherein the ON/Off switch is positioned to be activated for a limited period of time when the heating element lid is closed.

15. The ecofriendly diaper system according to claim 14, wherein the diaper container has a thermal insulator between the pair of heating elements and the storage space.
